(19) United States
(12) Patent Application Publication
(10) Pub. No.: US 2009/0162439 A1
(43) Pub. Date: Jun. 25, 2009

(54) SILK FIBROIN COATING

(75) Inventor: Andrea S. Gobin, Shepherdsville, KY (US)

Correspondence Address: VIKSNINS HARRIS & PADYS PLLP, P.O. BOX 111098, ST. PAUL, MN, 55111-1098, US

(73) Assignee: University of Louisville Research Foundation, Louisville, KY (US)

(21) Appl. No.: 12/341,461

(22) Filed: Dec. 22, 2008

Related U.S. Application Data
(60) Provisional application No. 61/016,450, filed on Dec. 22, 2007.

(51) Int. Cl. A01N 63/00; A01N 25/34; A01N 37/52; A01N 43/60; A61M 25/00; A01N 43/42; A01N 43/54

(52) U.S. Cl. 424/486; 514/636; 514/311; 514/275; 514/8; 514/254.1; 604/265; 424/94.61

(57) ABSTRACT

The present invention is directed to compositions and methods for preventing or inhibiting biofilm formation on a solid substrate comprising an antimicrobial agent and a biofilm-degrading enzyme embedded in a matrix material.

### RELATED APPLICATION

This application claims the benefit of priority of U.S. Provisional Application No. 61/016,450, filed Dec. 22, 2007. The entire content of this application is hereby incorporated herein by reference.

### BACKGROUND OF THE INVENTION

Catheter-related blood stream infections (CRBSI) are the leading cause of nosocomial blood stream infections and are associated with significant morbidity and mortality in critically ill patients. These infections represent a challenge to the treating physician because the bacteria exist on the catheter in a complex, community-like structure known as a biofilm. Biofilms are matrix-enclosed bacteria populations that are adherent to the surface of solid substrates, such as a catheter. Once a patient develops a CRBSI, removal of the catheter is necessary as part of the treatment. The need to remove the infected catheter and to place a new catheter is associated with increased risk for medical complications and increased healthcare costs.

CRBSIs typically develop as a result of bacterial adherence and bacteria colonization in a complex architecture, or biofilm, on catheter surfaces with the extent and location of biofilm formation depending on the duration of catheterization. Current technology for the prevention of CRBSIs includes silver ion impregnated catheters, antimicrobial impregnated catheters and dressings, aseptic catheter hubs, and antimicrobial/anticoagulant flush solutions. These strategies all reduce the risk of CRBSIs for short-term catheter use; however, they typically fail for long-term catheter use in chemotherapy or dialysis applications. Specifically, up to 60% of the new hemodialysis patients use a catheter for vascular access greater than 30 days but, infection, thrombosis, and central vein stenosis remain the primary causes of failure.

### SUMMARY OF THE INVENTION

The present invention provides a composition for preventing or inhibiting biofilm formation on a solid substrate. As used herein, the term “preventing or inhibiting” means that the amount of biofilm is reduced by any amount between 0-100%. The biofilm-prevention composition includes an antimicrobial agent and a biofilm-degrading enzyme embedded in a matrix material. In certain embodiments, the antimicrobial agent is chlorhexidine, a quinolone, silver sulfadiazine, vancomycin, Chlorhexidine salts (diacetate or digluconate), or Rifampin. In certain embodiments, the biofilm-degrading enzyme is an exopolysaccharide-degrading or glycolaminoglycan-degrading enzyme. In certain embodiments, the biofilm-degrading enzyme is N-acetylglucosaminidase or Dispersin B. In certain embodiments, the biofilm-degrading enzyme and/or the antimicrobial agent is encapsulated in chitosan. In certain embodiments, the matrix is silk fibroin, chitosan, polyethylene glycol, poly-vinyl alcohol, or a blend of one or more of these materials. In certain embodiments, the biofilm is formed by S. epidermidis, S. aureus, Candida albicans, Pseudomonas aeruginosa, Klebsiella pneumoniae, Enterococcus faecalis, and/or Corynebacterium. In certain embodiments, the silk fibroin is silkworm silk, such as from Bombyx mori, or a wild silkworm, such as Antheraea pernyi. In certain embodiments, the silk fibroin is spider silk, such as from Nephila clavipes. In certain embodiments, the silk fibroin is a genetically engineered silk.

The present invention provides an article of manufacture comprising a solid substrate coated with the composition described hereinabove. In certain embodiments, the solid substrate is a catheter, such as a peripheral intravenous catheter, a central venous catheter, or a urinary catheter, or a catheter hub, or a catheter port. In certain embodiments, the solid substrate is a non-degradable implant, such as a joint implant (knee, hip, ankle, etc.), a bone pin, or a prosthetic heart valve.

The present invention provides a method for producing a biofilm-inhibited article of manufacture composition by (a) contacting a silk fibroin solution with an antimicrobial agent and a biofilm-degrading enzyme to form a biofilm-inhibiting solution, and (b) coating a solid substrate with the biofilm-inhibiting solution. In certain embodiments, the antimicrobial agent is chlorhexidine or a quinolone. In certain embodiments, the biofilm-degrading enzyme is a polysaccharide or glycolaminoglycan degrading enzyme. In certain embodiments, the biofilm-degrading enzyme is N-acetylglucosaminidase. In certain embodiments, the matrix is silk fibroin chitosan, polyethylene glycol, poly-vinyl alcohol, or a blend of one or more of these materials. In certain embodiments, the biofilm is formed by S. epidermidis, S. aureus, Candida albicans, Pseudomonas aeruginosa, Klebsiella pneumoniae, and/or Enterococcus faecalis.

The silk solution generally will stick to the surface after drying. In case adherence is not sufficient, the pretreated catheter surfaces (pretreated for hydrophobicity) can have the layer stripped before coating with the biofilm-inhibiting solution. Other methods to increase surface hydrophobicity include chemical modification of surface to increase concentration of hydrophobic and/or non-polar molecules.

The present invention provides a method for producing a biofilm-inhibited article of manufacture composition by (a) contacting a silk fibroin solution with an antimicrobial agent and a biofilm-degrading enzyme to form a biofilm-inhibiting solution, and (b) forming a silk fibroin article comprising the biofilm-inhibiting solution. In certain embodiments, the silk fibroin article is a thread, fiber, film, foam, mesh, hydrogel, three-dimensional scaffold, tablet filling material, tablet coating, or microsphere. In certain embodiments, the silk fibroin solution is obtained from a solution containing a dissolved silkworm silk. In certain embodiments, the silkworm silk is obtained from Bombyx mori. In certain embodiments, the silk fibroin solution is obtained from a solution containing a dissolved spider silk. In certain embodiments, the spider silk is obtained from Nephila clavipes. In certain embodiments, the silk fibroin solution is obtained from a solution containing a genetically engineered silk.

### BRIEF DESCRIPTION OF THE FIGURES

FIG. 1. Schematic of silk fibroin coating delivering chlorhexidine and chitosan-NAG particles. CHX will kill the bacteria. In microenvironments where pH is low, chitosan will swell and deliver NAG to degrade biofilm.

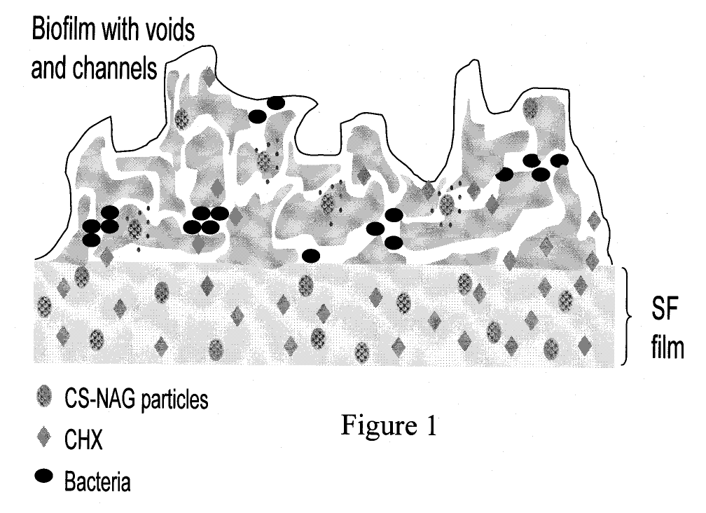

### DETAILED DESCRIPTION OF THE INVENTION

Catheter-Related Blood Stream Infections (CRBSI)

Biofilms are formed on catheters in two phases. The first phase is an initial adherence of the bacteria to the surface of the catheter. The second phase involves intercellular adhesion and the formation of micro-colonies with the production of a complex biofilm architecture. Since CRBSIs are extremely difficult to treat, much emphasis has been placed in prevention strategies. One important prevention strategy that is currently used in clinical practice is to impregnate the catheter with antibiotics to prevent the first phase of biofilm formation by preventing the adherence of bacteria to the catheter. This strategy does not entirely address bacterial pathogenesis, and is only effective for short-term catheters application (6-14 days). This strategy typically fails in long-term catheter applications such as cancer patient chemotherapy or hemodialysis in renal failure patients.

In an attempt to improve current CRBSI prevention strategies, the inventors developed a catheter that is treated with (1) antimicrobics to prevent the initial phase of biofilm formation; and (2) biofilm-degrading enzymes to inhibit the second phase of biofilm formation. These antimicrobics and exopolysaccharide-degrading enzymes are incorporated in silk fibroin matrix to sustain a proper dose of these agents over a prolonged period of time. Studies on silk fibroin as a drug delivery vehicle has shown enhanced drug delivery with targeting capabilities. A catheter coating derived from silk fibroin (SF) delivering both antimicrobial agents and biofilm-degrading enzymes reduced the risk of CRBSIs in long-term catheter applications.

Biofilms

A biofilm is a layer composed of water (up to 97%), polysaccharides (1-2%) and proteins (<1%) in which microorganisms (2-5%) encase themselves. Though biofilms are not required for initial bacteria adhesion, biofilm formation is essential for bacteria colonization and retention in a fluidic environment. In addition to providing microorganisms protection against host defenses and slowing antimicrobial therapy, biofilms can also serve as a nutrient source for the cells. The architecture of biofilm matrices elucidated via confocal scanning laser microscopy is structurally complex and heterogeneous. Studies show that biofilms are comprised of aggregates of microbial cells within the matrix of negatively charged exopolysaccharide (EPS) with interstitial voids and channels which separate the microcolonies. These channels permit the flow of nutrients, enzymes, metabolites, waste products and other solutes throughout the biofilm. However, the differences in colony densities produce microscale gradients in nutrient concentrations and metabolic products. Local accumulation of acidic waste products can lead to microenvironments of pH differences.

The most commonly isolated bacteria in catheter biofilms are S. epidermidis, S. aureus, Candida albicans, Pseudomonas aeruginosa, Klebsiella pneumoniae, and Enterococcus faecalis. In S. aureus and S. epidermidis biofilms, the important EPS component identified for intercellular adhesion and required for biofilm adhesion is polysaccharide intercellular adhesin (PIA) or capsular polysaccharide-adhesin (PS/A). Both adhesins have been found to have similar chemical structures, poly-β(1-6)-N-acetylglucosamine (PNAG), with differences in molecular size, biophysical properties, the degree of N-acetylation, the degree of O-succinylation, and immunogenecity. β-N-acetylglucosaminidase (NAG) is a lysosomal enzyme in mammalian cells, which liberates terminal β-linked N-acetylglucosamine and N-acetylgalactosamine from a variety of substrates.

Antimicrobial resistance is thought to occur via three mechanisms: 1) Poor penetration of the antimicrobial drugs into the bacterial biofilms, thus the drug is either adsorbed or deactivated before it reaches the organisms; 2) Microenvironments within the biofilm created by the microorganisms' metabolism causing pockets of pH gradients, thus deactivating the drug or causing the bacteria to enter a non-growing phase, hence, protecting it from the drug; and 3) Phenotype resistance can occur in a sub-population of the organisms, though this is usually less than 1% of the original population. The bacteria within the biofilms can evade host immune responses and withstand antimicrobial therapy. However, when bacteria are dispersed from a biofilm they usually become rapidly susceptible to antibiotics.

The best studied antimicrobial catheters are those that are impregnated with a combination of either chlorhexidine gluconate and silver sulfadiazine, or minocycline and rifampin. It was shown that short-term use of chlorhexidine-silver sulfadiazine impregnated catheters (<10 days) reduced CRBSIs. However, with longer periods of time there was reduced antimicrobial activity and efficacy. Minocycline-rifampin impregnated catheters on the other hand were found to be associated with lower incidence of CRBSIs. However, in a study that investigated infection incidence with similar catheterization duration, it was uncertain if the reduced infection rates were due to increased amounts of active ingredients owing to the extent of catheter impregnation. Minocycline-rifampin catheters were both extraluminally and intraluminally impregnated, whereas, chlorhexidine-silver sulfadiazine impregnated catheters were only extraluminally impregnated. Other therapies include daily flushing with antimicrobial and anticoagulant solutions. Unfortunately, these systems only address the physiology of microorganisms and not biofilm formation directly, which is a major disadvantage. The present inventors examined a new technology which provides a coating that can control delivery of antimicrobial agents and biofilm-degrading enzymes to reduce CRBSIs via bacterial pathogenesis and biofilm composition.

Silk Fibroin (SF)

Silk, as the term is generally known in the art, means a filamentous fiber product secreted by an organism such as a silkworm or spider. Silks produced from insects such as Bombyx mori silkworms, and from the glands of spiders, typically Nephilia clavipes, are well-studied forms of the material. Many natural variants of silk, however, exist in nature. Fibroin is produced and secreted by a silkworm's two silk glands. The Bombyx mori silkworm produces a silk fiber (known as a “bave”) and uses the fiber to build its cocoon. The bave includes two fibroin filaments or “broins,” which are surrounded with a coating of gum, known as sericin. The sericin is the silk fibroin filament that possesses significant mechanical integrity. When silk fibers are harvested for producing yarns or textiles, including sutures, a plurality of fibers can be aligned together, and the sericin is partially dissolved and then resolidified to create a larger silk fiber structure having more than two broins mutually embedded in a sericin coating. The unique mechanical properties of reprocessed silk such as fibroin and its biocompatibility make the silk fibers especially attractive for use in biotechnological materials and medical applications. Silk provides an important set of material options for biomaterials and tissue engineering because of the impressive mechanical properties, biocompatibility and biodegradability.

Silk Fibroin (SF) is a fibrous protein comprised of two chains: a heavy chain, approximately 325 kDa, and a light chain, approximately 25 kDa. The two chains are linked by a single disulfide bridge. The individual fibroin fibers are bound together by a sericin coating, which, if removed prior to use, eliminates the thrombogenic and inflammatory responses of raw silk. SF's molecular structure provides unique mechanical properties that includes a high tensile strength together with flexibility. SF is an attractive material for biomedical applications due to its permeability to oxygen and water, cell adhesion properties, relatively low thrombogenicity, low inflammatory response, strong association with polysaccharides, and protease susceptibility. SF produced by the B mori has been extensively investigated and used in studies such as enzyme immobilization for biophotosensors, oxygen-permeable membranes for biomaterial applications, coatings for drug delivery, and as scaffold material for tissue engineering applications. The strong affinity to polysaccharides is exploited in the present research as a substrate to not only delivery the antimicrobial and the biofilm-degrading enzyme, but also to localize bacteria adhesion and biofilm formation.

The antimicrobial is delivered via diffusion directly from the SF coating. The biofilm-degrading enzyme in some embodiments is encapsulated in chitosan particles, which is then entrapped in the SF coating. As used herein, the term “particles includes both nanoscale and microscale particles. Thus, release of the enzyme depends not only on diffusion of the particles from the coating, but also release from the particles once they are in the correct micro-environment within the biofilm. Fibroin has a strong affinity to polysaccharides, which make up a large percentage of biofilms.

Chitosan (CS)

Chitosan (CS) is a partially deactylated form of chitin, an abundant polysaccharide derived from crustacean shells. It is structurally similar to glycosaminoglycans. The molecular weight of chitosan can vary from 50 kDa to >1000 kDa depending on the source and preparation procedure. CS is a cationic polymer whose crystallinity is a function of the degree of deacetylation. The deacetylation degree can range from 50 to 90%. Chitosan's excellent potential as a biomaterial derives from its cationic and high-charge density properties, which allow chitosan to form insoluble ionic complexes with a variety of anionic polymers. CS has also been shown to have wound healing properties, is nontoxic, and has minimal foreign body response with accelerated angiogenesis. To date, CS has been used in the medical field as wound dressings drug delivery systems and space filling implants. CS in the present invention is used to modulate delivery of biofilm-degrading enzymes through interaction with SF coatings and swelling properties within biofilm microenvironments. This combination of antimicrobial agents with biofilm-degrading enzymes in a controlled delivery system enables a unique method to reduce CRBSIs.

Encapsulation methods include emulsion and iontropic gelation. The gelation process occurs because of the inter- and intramolecular linkages created between polyanions such as tripolyphosphate (TPP) and the positively charged amino groups of the chitosan. The enzyme is mixed with the chitosan before addition of TPP. The particles spontaneously form. Size and distribution of particles is dependent on CS and TPP concentration and volumes.

Other Matrix Materials

The matrix material can be fabricated from biological polymers or from synthetic polymers, and combinations thereof. Suitable synthetic polymers include, for example, polyamides (e.g., nylon), polyesters (e.g., polyethylene teraphthalate), polyacetals/polyketals, polystyrenes, polyacrylates, vinyl polymers (e.g., polyethylene, polytetrafluoroethylene, polypropylene and polyvinyl chloride), polycarbonates, polyurethanes, poly dimethyl siloxanes, cellulose acetates, polymethyl methacrylates, polyether ether ketones, ethylene vinyl acetates, polysulfones, nitrocelluloses, similar copolymers and mixtures thereof. Bioresorbable synthetic polymers can also be used such as dextran, hydroxyethyl starch, derivatives of gelatin, polyvinylpyrrolidone, polyvinyl alcohol, poly[N-(2-hydroxypropyl)methacrylamide], poly(hydroxy acids), poly(epsilon-caprolactone), polylactic acid, polyglycolic acid, poly(dimethyl glycolic acid), poly(hydroxy butyrate), and similar copolymers. Suitable biological polymers include, without limitation, collagen, elastin, keratin, gelatin, polyamino acids, cat gut sutures, polysaccharides (e.g., cellulose and starch) and mixtures thereof. Biological polymers generally are bioresorbable. Purified biological polymers can be appropriately formed into a polymer material for further processing into fibers.

Solid Substrates

The present technology is a coating prepared from a matrix material, such as silk fibroin, that delivers antibiotics and biofilm-degrading enzymes that prevent and treat blood stream infections. The coating may be placed onto various solid substrates, such as a catheter (e.g., a peripheral intravenous catheter, a central venous catheter, or a urinary catheter, or a catheter hub, or a catheter port) or a non-degradable implant (e.g., such as a joint implant (knee, hip, ankle, etc.), a bone pin, or a prosthetic heart valve). In particular, the coating may be placed on vascular catheters that are typically used for long periods of time, such as with dialysis and cancer patients. The coatings are also used to deliver antibiotics and biofilm-degrading enzymes from orthopedic and cardiovascular devices for the prevention of bloodstream infections, and to delivery antibiotics and biofilm-degrading enzymes from urological catheters for the prevention of infections.

For example, the inventors coated polyurethane catheters with silk fibroin containing chlorohexidine and beta-N-acetylglucosaminidase, antibiotic and enzyme, respectively. The micro-organisms we are using for the studies are Staphylococci epidermis and Staphylococci aureus.

The silk fibroin provides several functions: 1) drug and enzyme carrier; 2) surface for attachment of bacteria and formation of biofilms; 3) induce more efficient release kinetics of the drugs and enzymes. Antibiotics are currently impregnated into catheter materials and work well for short periods (up to 14 days).

Antimicrobial Agents

The variety of different antimicrobial agents that can be used in the present invention is vast. Examples of anti-bacterial compounds suitable for use in the present invention include, but are not limited to, 4-sulfanilamidosalicylic acid, acediasulfone, amfenac, amoxicillin, ampicillin, apalcillin, apicycline, aspoxicillin, aztreonam, bambermycin(s), biapenem, carbenicillin, carumonam, cefadroxil, cefamandole, cefatrizine, cefbuperazone, cefclidin, cefdinir, cefditoren, cefepime, cefetamet, cefixime, cefinenoxime, cefminox, cefodizime, cefonicid, cefoperazone, ceforanide, cefotaxime, cefotetan, cefotiam, cefozopran, cefpimizole, cefpiramide, cefpirome, cefprozil, cefroxadine, ceftazidime, cefteram, ceftibuten, ceftriaxone, cefuzonam, cephalexin, cephaloglycin, cephalosporin C, cephradine, ciprofloxacin, clinafloxacin, cyclacillin, enoxacin, epicillin, flomoxef, grepafloxacin, hetacillin, imipenem, lomefloxacin, lymecycline, meropenem, moxalactam, mupirocin, nadifloxacin, norfloxacin, panipenem, pazufloxacin, penicillin N, pipemidic acid, quinacillin, ritipenem, salazosulfadimidine, sparfloxacin, succisulfone, sulfachrysoidine, sulfaloxic acid, teicoplanin, temafloxacin, temocillin, ticarcillin, tigemonam, tosufloxacin, trovafloxacin, vancomycin, and the like.

In certain embodiments, the antimicrobial agent can be chlorhexidine, a quinolone, silver sulfadiazine, vancomycin, Chlorhexidine salts (diacetate or digluconate), or Rifampin.

Biofilm-Degrading Enzymes

Many biofilm-degrading enzymes are known in the art. In certain embodiments, the biofilm-degrading enzyme is an exopolysaccharide-degrading or glycolaminoglycan-degrading enzyme. In certain embodiments, the biofilm-degrading enzyme is N-acetylglucosaminidase or Dispersin B.

Additives

In certain embodiments of the invention, the compositions may contain one or more additive components, which may be essential to the formation or existence of the formulation or may serve an auxiliary or secondary function, such as to homogenize the formulation. The additive of the present invention is non-polymeric in nature that can be incorporated into the present compositions to alter the mechanical or physical properties of the composition, such as viscosity, degree of cross-linking (in the case of hydrogels), degree of bioadhesion, release kinetics of a bioactive agent, or to facilitate some in situ reaction.

In situ reactions may be facilitated by the addition of pH adjusters. pH adjusters may take the form of acids, bases or buffers. Suitable acids include acetic acid, hydrochloric acid and benzoic acid. Suitable bases include sodium hydroxide, triethylamine. Various buffers based on phosphate, lactate and carbonate salts may also be employed to obtain pH values within the compositions, or parts of the composition in the range of 2 to 11.

For administration to a human or other mammal, the treatment compositions will often be sterilized or formulated to contain one or more preservatives for incorporation into pharmaceutical, cosmetic or veterinary formulations. These treatment compositions can be sterilized by conventional, well-known sterilization techniques, e.g., boiling or pasteurization when the drug is thermally stable. For drugs that are not thermally stable, then irradiation and/or a preservative may be utilized to provide a sterile composition. A preservative may be incorporated into a formulation of the present invention in an amount effective for inhibiting the growth of microbes, such as bacteria, yeast and molds. Any conventional preservative against microbial growth can be employed so long as it is pharmaceutically acceptable, is unreactive with the drug(s) contained in the formulation, and is non-irritating or non-sensitizing to human skin. Exemplary preservatives include antimicrobial aromatic alcohols, such as benzyl alcohol, phenoxyethanol, phenethyl alcohol, and the like, and esters of parahydroxybenzoic acid commonly referred to as paraben compounds, such as methyl, ethyl, propyl, and butyl esters of parahydroxybenzoic acid and the like. The amount of preservative is typically not more than about two weight percent, based on the total weight of the formulation.

The described compositions may include one or more additives, such as, for example, fragrances, including pharmaceutically acceptable perfumes; excipients for providing texture (e.g., abrasives or microabrasives); and excipients for providing a cooling or heating sensation (e.g., camphor). Tonicity may be adjusted by the inclusion of buffer salts, sodium chloride, or non-ionic species such as dextrose.

Methods of Manufacture

The present invention provides methods for preparation of silk-based drug delivery systems are described. In particular, the drug delivery system includes a composition for preventing or inhibiting biofilm formation in vivo. In general, a silk fibroin solution is combined with antimicrobial agent and a biofilm-degrading enzyme to form a silk fibroin article. In one embodiment a catheter coating is derived from silk fibroin (SF), the antimicrobial agent is chlorhexidine (CHX), and the biofilm-degrading enzyme is β-N-acetylglucosaminidase.

As used herein, the term “fibroin” includes silkworm fibroin and insect or spider silk protein. In certain embodiments, fibroin is obtained from a solution containing a dissolved silkworm silk or spider silk. The silkworm silk protein is obtained, for example, from Bombyx mori, and the spider silk is obtained from Nephila clavipes. In the alternative (or in addition), the silk proteins suitable for use in the present invention can be obtained from a solution containing a genetically engineered silk, such as from bacteria, yeast, mammalian cells, transgenic animals or transgenic plants. See, for example, WO 97/08315 and U.S. Pat. No. 5,245,012.

The silk fibroin solution can be prepared by any conventional method known to one skilled in the art. For example, B. mori cocoons are boiled for about 30 minutes in an aqueous solution. Preferably, the aqueous solution is about 0.02M Na₂CO₃. The cocoons are rinsed, for example, with water to extract the sericin proteins and the extracted silk is dissolved in an aqueous salt solution. Salts useful for this purpose include lithium bromide, lithium thiocyanate, calcium nitrate or other chemicals capable of solubilizing silk. Preferably, the extracted silk is dissolved in about 9-12 M LiBr solution. The salt is consequently removed using, for example, dialysis. If necessary, the solution can then be concentrated using, for example, dialysis against a hygroscopic polymer, for example, PEG, a polyethylene oxide, amylose or sericin. In certain embodiments, the PEG is of a molecular weight of 8,000-10,000 g/mol and has a concentration of 25-50%. Dialysis is performed for a time period sufficient to result in a final concentration of aqueous silk solution between 10-30%, such as for about 2-12 hours. Alternatively, the silk fibroin solution can be produced using organic solvents.

In accordance with the present invention, the silk fibroin solutions contain at least one antimicrobial agent and at least one biofilm-degrading enzyme. The silk fibroin solution is contacted with an antimicrobial agent and a biofilm-degrading enzyme prior to forming the fibroin article, e.g. a fiber, mesh, scaffold, or loaded into the article after it is formed. For loading after formation, silk assembly is used to control hydrophilic/hydrophobic partitioning and the adsorption of phase separation of the antimicrobial agent and the biofilm-degrading enzyme. The material can also be loaded by entrapping the antimicrobial agent and/or the biofilm-degrading enzyme in the silk.

Silk formulations containing bioactive materials may be formulated by mixing one or more antimicrobial agents and biofilm-degrading enzymes with the silk solution used to make the article. Alternatively, an antimicrobial agent and/or biofilm-degrading enzyme can be coated onto the pre-formed silk fibroin article, such as with a pharmaceutically acceptable carrier. Any pharmaceutical carrier can be used that does not dissolve the silk material. The antimicrobial agents and biofilm-degrading enzymes may be present as a liquid, a finely divided solid, or any other appropriate physical form.

The above described silk fibroin solution, which contains at least one antimicrobial agent and at least one biofilm-degrading enzyme, is next processed into a thread, fiber, film, mesh, hydrogel, three-dimensional scaffold, tablet filling material, tablet coating, or microsphere. Methods for generating such are well known in the art.

Silk films can be produced by preparing the concentrated aqueous silk fibroin solution and casting the solution. The film can be contacted with water or water vapor, in the absence of alcohol. The film can then be drawn or stretched mono-axially or biaxially. The stretching of a silk blend film induces molecular alignment of the film and thereby improves the mechanical properties of the film.

The present invention additionally provides a non-woven network of fibers comprising a pharmaceutical formulation of the present invention. The fiber may also be formed into yarns and fabrics including for example, woven or weaved fabrics. The fibroin silk article of the present invention may also be coated onto various shaped articles including biomedical devices (e.g., stents), and silk or other fibers, including fragments of such fibers.

The silk fibroin articles described herein can be further modified after fabrication. For example, the scaffolds can be coated with additives, such as bioactive substances that function as receptors or chemoattractors for a desired population of cells. The coating can be applied through absorption or chemical bonding.

Additives suitable for use with the present invention include biologically or pharmaceutically active compounds. Examples of biologically active compounds include, but are not limited to, cell attachment mediators, such as collagen, elastin, fibronectin, vitronectin, laminin, proteoglycans, or peptides containing known integrin binding domains, e.g., “RGD” integrin binding sequence, or variations thereof, that are known to affect cellular attachment; biologically active ligands; and substances that enhance or exclude particular varieties of cellular or tissue in-growth.

The silk-based system of the present invention may comprise a plurality (i.e., layers) of silk fibroin articles. For example, each layer may have different solutions of fibroin (concentrations, drugs).

The following examples are intended to illustrate particular embodiments, and not limit the scope, of the invention. Those of ordinary skill in the art will readily recognize that additional embodiments are encompassed by the invention. It will be clear that the invention may be practiced otherwise than as particularly described in the foregoing description and examples. Numerous modifications and variations of the present invention are possible in light of the above teachings and, therefore, are within the scope of the appended claims.

### EXAMPLE 1

SF can be used as a drug delivery vehicle by exploiting the muco-polysaccharide adhesive properties of SF to enhance targeted delivery of the drug. SF was used to coat 1,2 dimyristolsn-glycero-3phosphocholine (DMPC) liposomes that contained the drug emodin to treat keloids. Emodin (3-methyl-1,6,8,trihydroxyanthran-quinone) is a relatively selective receptor tyrosine kinase (RTK) inhibitor that naturally occurs. Keloids are chronic dermal wounds resulting from a cutaneous injury caused by surgery or inflammation. They are characterized as raised pathological scars, causing pain and persistent itching, and are considered to be benign dermal tumors. Chronic dermal wounds consist of fibroblasts that overproduce collagen and chondroitin sulfate, have high contractile activity, high levels of secreted cytokines, and are similar to tumors in overexpression of RTK, a transmembrane receptor that binds to growth factors such as fibroblast growth factor (FGF). It was observed that the drug release kinetics changed due to the structural interactions between the SF and the liposome lamellae. Furthermore, targeted delivery was enhanced by the SF association with the muco-polysaccharides, which are over-expressed by the keloid cells.

These studies show that increased association between the biofilm exopolysaccharides and SF can provide an environment to enhance the delivery of antibiotics and enzymes by localizing initial adhesion of the organism and biofilm formation.

### EXAMPLE 2

The inventors developed a SF coating on vascular catheters to deliver both antimicrobial agents and proteolytic enzymes that work synergistically, at the surface interface to prevent or treat bloodstream infections caused by S. epidermidis or S. aureus.

Release Kinetics of the Antimicrobial Drugs and Enzymes from the SF Films

Chlorhexidine (CHX) is a widely used antimicrobial agent that possesses a broad spectrum of activity against bacteria. It is delivered directly from the SF film via diffusion to reduce bacteria viability. β-N-acetylglucosaminidase (NAG; Enzyme Commission Number 3.2.1.52; Sigma®-Aldrich®) is a lysosomal enzyme that hydrolyzes the terminal non-reducing N-acetyl β-glucosaminides. NAG is used to degrade or disrupt the Staphylococcal biofilms, which contain a β(1-6) linked N-acetylglucosamine homoglycan. NAG is encapsulated in chitosan microspheres, which in turn is entrapped in the SF film. The reasoning for this is twofold: 1) it slows the diffusion kinetics of NAG so that it is released during biofilm formation, and 2) NAG is only released in microenvironments in which it is active. Due to the molecular interaction between the SF and CS, CS particles are released from the SF films at a slower rate. In addition, NAG is released from the CS particles and most active when it reaches a microenvironment with low pH, such as less than about pH 6. This is due to the swelling behavior of CS in acidic environments.

SF Fiber Dissolution and Film Preparation

The sericin coating of virgin silk is removed with 0.25% w/v sodium lauryl sulfate (SDS; Sigma®-Aldrich®, St Louis, Mo., USA) and 0.25% w/v sodium carbonate (Sigma®-Aldrich®) in boiling water for 1 hour. The degummed fibroin is washed in boiling water for 1 hour and rinsed again in distilled water to remove the remaining sericin and surfactants. Dried SF is dissolved in calcium nitrate tetrahydrate (Ca(NO₃)₂.4H₂O; Fisher Scientific, Pittsburgh, Pa., USA) methanol solution. To achieve this, calcium nitrate tetrahydrate is dissolved in methanol at 1:4:2 molar ratio (Ca:H₂O:MeOH) at room temperature for 1 hour while stirring. The solution temperature is raised to 65° C. and SF is added to a final concentration of 10% w/v. The SF is allowed to dissolve for 4 hours with continuous stirring at 65° C. Dissolved SF is stored at 4° C. until needed. Aqueous SF is obtained after the dissolution mixture is dialyzed against deionized water for 4 days with a change of water each day (6000-8000 Da MWCO; Fisher Scientific, Pittsburgh, Pa., USA). Aqueous SF is stored at 4° C. until used. Final protein concentration is measured using bicinchoninic acid (BCA) protein assay (Pierce, Rockford, Ill.). Films are prepared by pipetting solution (either methanol based or aqueous) onto a glass slide, and drying at room temperature for 24 hrs. To control film thickness, a spin coater (Headway, Garland, Tex.) is used to cast the films. To insolubilize the SF, films are immersed in 50% v/v methanol and water for 15 minutes. SF films are stored hydrated at 4° C. until used.

Antimicrobial Agent in SF Films Preparation

Chlorhexidine (CHX; Sigma®-Aldrich®, St. Louis, Mo.) is widely used as an antiseptic and for catheter impregnation; thus, CHX is used as the antimicrobial agent in this study. CHX is dissolved in methanol (2% or 4% w/v) then mixed with varying protein concentrations of the methanol-based SF solution. SF-CHX films are cast as described above. Dried films are insolubilized by immersion in 50% v/v methanol and water (crystallization solution) for 15 minutes. CHX loading capacity and efficiency of the SF films is evaluated by measuring free CHX in the crystallization solution by UV spectrophotometry at λ=231 nm. Loading capacity (LC) is defined by Equation D1 (Loading Capacity of CHX in SF film) and efficiency is defined by Equation D2.

$$\begin{matrix}Loading Capacity = \frac{Total amount CHX - Free Amount CHX}{Volume of SF film ( dry )} \times 100 & Equation D1 \\ Loading Efficiency = \frac{Total amount CHX - Free Amount CHX}{Total amount of CHX} \times 100 & Equation D2\end{matrix}$$

Chitosan Particles Preparation

CS particles are obtained by inducing the gelation of a CS solution by interaction with a polyanion pentasodium tripolyphosphate (TPP; Sigma®-Aldrich®). CS solution is prepared by dissolving high molecular weight chitosan (>75% deacetylation: Sigma®-Aldrich®) in 2% acetic acid at various concentrations. TPP is dissolved in deionized water at the same concentrations of CS. Variable volume of TPP is added to the CS solution and mixed at room temperature. Particles spontaneously form and can be concentrated by centrifugation at 16,000×g in a 10 82 l glycerol bed for 30 minutes. Supernatants are aspirated and particles resuspended in phosphate buffered saline (PBS, pH 7.4) for transmission electron microscopy (TEM; FEI Tecnai™, Hillsboro, Oreg.) and swelling behavior studies. Swelling behavior is characterized by measuring weight difference of particles in different pH solutions (2 and 5). The ratio of the weight difference (neutral−acidic) to the total weight at neutral pH allows for the estimation of the swelling capabilities of the particles.

Chitosan-NAG Particles Preparation

CS-NAG particles are prepared similarly to the above described method. NAG is added to the dissolved CS solution at various concentrations. Particles form after the addition of the TPP solution. Similar loading capacity and efficiency studies (described above) are performed by measuring free NAG by UV spectrophotometry at λ=280 nm.

In Vitro Release Profile Evaluation

Release profiles are determined for CHX release from SF films, NAG release from CS particles, CS particle release for SF films and NAG release from particles after release from SF films. In addition, both NAG and CHX simultaneous release profiles from SF films are determined. All studies are conducted in both a static environment and dynamic flow environment at 37° C. Static tests are conducted in vials that provide a maximum surface-to-volume ratio. Dynamic tests involve films layered between glass slides and a continuous flow pump (Bamant Manostat, Barrington, Ill.) that circulate saline solution at approximately 0.5 mm/sec. Films are incubated in saline at 37° C. throughout the study. Supernatant samples are taken at predetermined time points, ranging from hours, to days to weeks. Samples are analyzed for drug or enzyme content and their respective concentrations. CHX is measured spectrophotometrically at 231 nm. N-acetylglucosaminidase release and activity is measured using a colorimetric substrate at 405 nm (β-N-acetylglucosaminidase assay kit; Sigma®-Aldrich®). Optical absorbencies are converted to concentrations using standard curves of known concentrations. A minimum of 3 samples are taken at each time point. Drug and enzyme release mechanisms and kinetics are described by fitting the data to the power law equation (Equation D3).

$$\frac{{M}_{t}}{{M}_{∞}} = {kt}^{n} \qquad Equation D3$$

where Mₜ is amount of drug released at time t; M₂₈ is total amount of drug release at infinite time; k is release constant related to structural and geometric properties; and n is release exponent indicating type of release mechanism.

All studies are conducted in at least triplicate and are statistically analyzed using GraphPad software (San Diego, Calif.) with a p-value≦0.05 considered significantly different.

CHX and NAG produce different release profiles due to the different delivery modes. CHX is released via diffusion mechanisms and can be varied by changing the SF concentration or layering of different SF-CHX films to provide multiple profile release curves. NAG is released via diffusion and swelling mechanisms due to the SF and CS interactions and behavior. Initially CS-NAG particles diffuse from the SF film. Consequently, in the correct pH microenvironment there is swelling of the chitosan with burst release of the NAG. With controlled delivery of both CHX and NAG, bacteria viability and biofilm formation is reduced simultaneously. In addition, it is believed that if CS-NAG particles are released with no biofilm formation, NAG is not released due to pH stability and hence, no swelling behavior will occur in the CS.

Alternative Approaches

An alternative approach is to use a different delivery vehicle such as poly (glycolic-lactic) acid (PGLA) which releases the enzyme due to degradation via hydrolysis. This approach allows quick delivery, based on the glycolic to lactic acid ratio, throughout the biofilm, but does not allow for controlled delivery to microenvironments where NAG would be active.

Another alternative is to use chlorhexidine salts, such as chlorhexidine digluconate, instead of the SF films.

### EXAMPLE 3

The SF film preparations described in Example 2 above are accomplished with glass substrates to which the SF adheres. In order to assess CHX and NAG release from catheter coatings, SF adhesion to common catheter materials is analyzed. In addition, techniques are developed to coat catheters evenly both extraluminally and intraluminally.

Catheter Selection

Selected catheters for this study are 20 cm long, 7 French, triple-lumen, polyurethane intravenous catheters (Cook Inc., Bloomington, Ind.), as this is commonly used. Polyurethane sheets (PSI Urethanes, Inc. Austin, Tex.) with similar thickness and mechanical properties to that of the catheters are also obtained to provide flat uniform substrates to compare to the glass substrates and for mechanical adhesion testing.

SF Adhesion to Polyurethane

SF adhesion studies are performed on flat polyurethane substrates. Substrates are coated with SF as previously described using a spin coater to control the film thickness. Various SF concentrations and thicknesses are examined. After coating, SF is made insoluble with the crystallization solution. Multi-layered coatings are applied in one of two methods: 1) applying several coats, allowing them to dry between coatings, then crystallization by methanol immersion; or 2) each coated layer is applied, dried, and crystallized between layers. Mechanical tensile and compression tests are performed to assess if SF films slip, tear or buckle with substrate deformation. Uncoated substrates that have been treated in the crystallization solution are examined as a control. A minimum of 5 samples of each condition is examined. Coated substrates are imaged via scanning electron microscopy (SEM; Carl Ziess® LEO EVO40) with Extended Pressure (EP) range up to 3000 Pa, to enable the imaging of dynamic processes involving water in life science and material analysis applications. Thus, coated substrates are imaged both in a dry and hydrated state.

SF Coated Catheters

Catheters are coated both internally and externally. For external coatings, catheters are dipped into the SF solution at room temperature, placing a thin layer of film on the polyurethane. The coating is allowed to dry for 24 hours at room temperature. Internal coatings are applied in a similar fashion, except the solutions are pumped (syringe pump, KD Scientific Inc., Holliston, Mass.) through the lumen, followed by an inert gas, argon, to aid in drying as well as insure catheter patency. SF film coatings contain either CHX only, CS-NAG only, or an appropriate combination of the two. Control catheters consist of the SF coating only with no drug or enzyme.

SF adheres well to polyurethane due to the hydrophobicity of both materials. Due to the flexibility of SF, it withstands the mechanical forces typically seen with handling of catheters in clinical environments. Strong adhesion of the SF to the catheter provides a uniform delivery mode for the CS-NAG and CHX.

### EXAMPLE 4

The previous Examples characterize material properties and determine release profiles of the antimicrobial agent and enzyme. Upon release of the antimicrobial agent and biofilm-degrading enzyme, the activity and efficiency of these components are determined. This is accomplished by first determining the appropriate levels of antibiotics and enzyme to entrap in the SF coatings that result in growth inhibition of the microorganisms in vitro. Hence, the minimal inhibitory concentrations (MIC) that are most efficacious is determined. In addition, the ability of the microorganisms to adhere to the SF coatings is accomplished using a simulated biofilm producing model.

Microorganisms

S. epidermidis (35983, 12228) and S. aureus (10390, 33591) organisms are obtained either from American Type Culture Collection (ATCC; Manassas, Va.). Laboratory control strains are used to address issues of varying levels of susceptibility to antibiotics. Organisms are maintained on Mueller Hinton II Broth (MHB) and Mueller Hinton II Agar (MHA) or drug neutralizing agar (DE) and drug neutralizing broth (LTSB) (Fisher Scientific Co., Atlanta, Ga.). Biofilm production is verified by growth of black colonies on Congo red agar.

Culture Media

Mueller Hinton II Broth (cation adjusted) MHB, Mueller Hinton II agar (MHA), and drug neutralizing agar (DE) are available from Fisher Scientific Co. (Atlanta, Ga., USA). The composition of drug neutralizing broth (LTSB) is 3% tryptic soy broth, 0.5% proteose peptone, 0.1% tryptone, 0.5% sodium thiosulphate, 0.6% sodium oleate, 2% lecithin and 5% Tween 80.

Soaking Media

A proteinaceous medium containing 5% bovine adult serum (BAS) and 5% total parenteral nutrition fluid (TPN) in phosphate-buffered saline (PBS) is constituted. BAS and PBS are purchased from Sigma®, St. Louis, Mo.

Antibiotic Spectrum Studies

Zone-of-inhibition testing is performed to determine the antimicrobial spectrum and the appropriate concentrations of drug and protease released. MHA plates are seeded on their surface with 0.3 mL of overnight cultures of different bacteria diluted to 108 CFU/mL in 0.45% saline using a spectrophotometric colorimeter. The inoculums are verified by quantitative culture of the inoculums by performing serial dilutions in saline followed by replicate agar plate counts. Catheters are aseptically cut into 0.5 cm segments and embedded vertically in the MHA. A total of 12 segments are tested against each organism. After 24 and/or 48 hours of incubation at 37° C., the diameter of the zone-of-inhibition is measured with a caliper. Controls consist of SF-coated catheters without CHX or CS-NAG.

MIC and MBC (Minimum Bactericidal Concentration) Determinations

MIC is determined by a standardized microplate, tube dilution method in MHB. Briefly, two-fold serial dilutions of the drugs are prepared in 5 mL MHB. Stock solutions (10,000 and 2,000 mg/L) of all the drugs, made in the appropriate solvent, are diluted and added to MHB-containing microplate tubes, keeping solvents at a concentration of 2.5% v/v in all tubes, including the control. An overnight culture grown in MHB is diluted and added to all the tubes for a cell density of 10⁴ CFU/mL. The microplate tubes are incubated for 24 h at 37° C. and checked for turbidity by absorbance measurements. For MBC determination, 0.1 mL aliquots from all tubes showing no visible growth are plated out on DE agar and incubated for 24 h. MBC is defined as the lowest antimicrobial concentration that killed≧99.9% of the inoculum.

Susceptibility Testing

Determining changes in MIC and measuring zones-of-inhibition produced in catheter segments is performed to analyze susceptibility. MICs are compared against passaged strains and MICs are determined by the microplate tube dilution method described above. Zones-of-inhibition produced by the loaded SF coated catheters against the original and passaged isolates are determined Briefly, 0.5 cm segments of the catheters are embedded in MHA plates seeded with 0.3 mL of overnight culture (diluted to 1×10⁸ CFU/mL). After incubation at 37° C., the zones-of-inhibition, including the diameter of the catheters, are measured with a caliper. The catheters are tested before and after shaking on a rotary shaker at 100 rpm and 37° C. for 7 and 14 days in proteinaceous medium (1 mL/cm). The medium is changed daily.

Correlation Between MIC and Zone Size of Antibiotic and Enzymatic Catheters

Various passages of the isolated and cultured bacteria have different MICs to the antibiotics. The passages with high and low MICs are used for this study. The zones-of-inhibition of catheters pre-soaked, and 7 and 14 days post-soaking are determined using these strains as described above.

Biofilm Growth in Microplates

Biofilms are grown in microplates whose well bottoms have been coated with loaded SF films or control SF films. Inoculum aliquots are added to wells and incubated overnight. The plates are then rinsed with PBS. Biofilms are stained with crystal violet and optical density measurements are taken at λ=590 nm.

Bacterial Adherence Studies

Loaded SF coated catheters and controls (4 cm length) are soaked for 7 and 14 days in proteinaceous medium (1 mL/cm) and evaluated for in vitro adherence of test organisms. In order to facilitate biofilm production, the cultures are grown overnight, at 37° C., in MHB supplemented with 0.25% glucose and diluted 1:5 with fresh medium. The cultures are incubated again for 4-6 h, until the absorbance (λ=600 nm) is 0.25-0.3. The cultures are centrifuged and the cells washed twice in PBS. The cells are resuspended in PBS supplemented with 0.5% glucose (glucose is added as a substrate for viability and slime production) and sonicated for 1 min to obtain a uniform suspension. After diluting to a density of 1-2×10⁵ CFU/mL, four segments are suspended in 6 mL of this culture in a tube, and incubated at 37° C. After 24 h, the catheters are removed, rinsed twice in saline and 1 cm segments cut-off at both ends. The remaining pieces (2 cm in length) are suspended in 4 mL LTSB and sonicated in an ultrasonic bath at 40 kHz for 20 min. Aliquots of the LTSB extract (0.5 mL) are plated out on DE agar and incubated at 37° C. for 24 h. This quantitative culture is used to determine the bacterial adherence and is expressed as the number of colony-forming units found in each segment.

Evaluation of the Neutralizing Efficacy of Drug Inactivation Media

To avoid false-negative results due to carry-over of drugs to the subculture media, drug-inactivating media is used. The efficacy of the neutralizing media is tested by suspending 2 cm segments of control or loaded SF coated catheters in 4 mL of LTSB inoculated with an overnight culture of bacteria to yield a density of 1×10³ CFU/mL. These are sonicated at 40 kHz for 20 min. Aliquots of this suspension (0.5 mL) are plated out on MHA and DE plates and incubated at 37° C. for 24 h to determine the colony count (0.5 mL aliquot of LTSB, similarly inoculated but not sonicated, are used as the control).

Statistical Analysis

All studies are statistically analyzed using GraphPad software (San Diego, Calif.) with a p-value≦0.05 considered significantly different.

Chlorhexidine is already known to decrease bacteria viability. It is typically coated directly to catheter materials. However, its efficiency is usually short lived. Control delivery from silk fibroin films increases delivery time, thus reducing bacteria adhesion and colony formation due to reduced viability. Common protocols for degradation of biofilms to examine molecular structure include using high temperatures and acids. Since the present inventors are looking for a means to do this in vivo, those protocols are unacceptable. β-N-acetylglucosaminidase has not been reported in the literature to degrade biofilms.

It must be noted that, as used in this specification and the appended claims, the singular forms “a,” “an” and “the” include plural referents unless the content clearly dictates otherwise. Unless otherwise defined, all technical and scientific terms used herein have the same meaning as commonly understood by one of ordinary skill in the art.

All publications, patents and patent applications are incorporated herein by reference in their entirety. While in the foregoing specification this invention has been described in relation to certain preferred embodiments thereof, and many details have been set forth for purposes of illustration, it will be apparent to those skilled in the art that the invention is susceptible to additional embodiments and that certain of the details described herein may be varied considerably without departing from the basic principles of the invention.

What is claimed is:

1. A composition for preventing or inhibiting biofilm formation on a solid substrate comprising an antimicrobial agent and a biofilm-degrading enzyme embedded in a matrix material.

2. The composition of claim 1, wherein the antimicrobial agent is chlorhexidine, a quinolone, silver sulfadiazine, vancomycin, Chlorhexidine salts (diacetate or digluconate), or Rifampin.

3. The composition of claim 1, wherein the biofilm-degrading enzyme is an exopolysaccharide-degrading or glycolaminoglycan-degrading enzyme.

4. The composition of claim 1, wherein the biofilm-degrading enzyme is N-acetylglucosaminidase or Dispersin B.

5. The composition of claim 1, wherein the matrix is silk fibroin, chitosan, polyethylene glycol, poly-vinyl alcohol, or a blend of one or more of these materials.

6. The composition of claim 1, wherein the biofilm is formed by S. epidermidis, S. aureus, Candida albicans, Pseudomonas aeruginosa, Klebsiella pneumoniae, or Enterococcus faecalis and/or and/or Corynebacterium.

7. The composition of claim 5, wherein the silk fibroin is silkworm silk, spider silk, or genetically engineered silk.

8. The composition of claim 7, wherein the silk is obtained from Bombyx mori.

9. The composition of claim 7, wherein, the silk is obtained from Nephila clavipes.

10. The composition of claim 1, wherein the biofilm-degrading enzyme is encapsulated in chitosan.

11. An article of manufacture comprising a solid substrate coated with the composition of claim 1.

12. The article of claim 11, wherein the solid substrate is a catheter, a catheter hub, a catheter port, or a non-degradable implant.

13. A method for producing a biofilm-inhibited article of manufacture composition comprising:
(a) contacting a silk fibroin solution with an antimicrobial agent and a biofilm-degrading enzyme to form a biofilm-inhibiting solution, and
(b) forming a silk fibroin article comprising the biofilm-inhibiting solution or coating a solid substrate with the biofilm-inhibiting solution.

14. The method of claim 13, wherein the antimicrobial agent is chlorhexidine, a quinolone, silver sulfadiazine, vancomycin, Chlorhexidine salts (diacetate or digluconate), or Rifampin.

15. The method of claim 13, wherein the biofilm-degrading enzyme is a polysaccharide or glycolaminoglycan degrading enzyme.

16. The method of claim 13, wherein the biofilm-degrading enzyme is N-acetylglucosaminidase or Dispersin B.

17. The composition of claim 13, wherein the biofilm-degrading enzyme is encapsulated in chitosan.

18. The method of 13, wherein the matrix is silk fibroin, chitosan, polyethylene glycol, poly-vinyl alcohol, or a blend of one or more of these materials.

19. The method of 13, wherein the solid substrate is a catheter, a catheter hub, a catheter port, or a non-degradable implant.

20. The method of claim 13, wherein a silk fibroin article is formed and the silk fibroin article is a thread, fiber, film, foam, mesh, hydrogel, three-dimensional scaffold, tablet filling material, tablet coating, or microsphere.

21. The method of claim 13, wherein the silk fibroin solution is obtained from a solution containing a dissolved silkworm silk, dissolved spider silk, or a genetically engineered silk.

22. The method of claim 21, wherein the silk is obtained from Bombyx mori or from Nephila clavipes.
